SEQUENCE LISTING (10 sequences)

SEQ ID NO: 1 - DNA, 25 bp, Artificial Sequence
  Oligonucleotide forward primer used in RT-PCR for amplifying a region of OCT-4 gene
cttgctgcagaagtgggtggaggaa

SEQ ID NO: 2 - DNA, 21 bp, Artificial Sequence
  Oligonucleotide reverse primer used in RT-PCR for amplifying a region of OCT-4 gene
ctgcagtgtgggtttcgggca

SEQ ID NO: 3 - DNA, 20 bp, Artificial Sequence
  Oligonucleotide forward primer used in RT-PCR for amplifying a region of nestin gene
gagagggaggacaaagtccc

SEQ ID NO: 4 - DNA, 20 bp, Artificial Sequence
  Oligonucleotide reverse primer used in RT-PCR for amplifying a region of nestin gene
tccctcagagactagcgcat

SEQ ID NO: 5 - DNA, 20 bp, Artificial Sequence
  Oligonucleotide forward primer used in RT-PCR for amplifying a region of musashi-1 gene
gaatggacgccttcatgctg

SEQ ID NO: 6 - DNA, 20 bp, Artificial Sequence
  Oligonucleotide reverse primer used in RT-PCR for amplifying a region of musashi-1 gene
cgctgatgtaactgctgacc

SEQ ID NO: 7 - DNA, 18 bp, Artificial Sequence
  Oligonucleotide forward primer used in RT-PCR for amplifying a region of Sox-2 gene
cccccggcggcaatagca

SEQ ID NO: 8 - DNA, 20 bp, Artificial Sequence
  Oligonucleotide reverse primer used in RT-PCR for amplifying a region of Sox-2 gene
tcggcgccggggagatacat

SEQ ID NO: 9 - DNA, 23 bp, Artificial Sequence
  Oligonucleotide forward primer used in RT-PCR for amplifying a region of Rex-1 gene
gcgtacgcaaattaaagtccaga

SEQ ID NO: 10 - DNA, 25 bp, Artificial Sequence
  Oligonucleotide reverse primer used in RT-PCR for amplifying a region of Rex-1 gene
cagcatcctaaacagctcgcagaat

(19) United States
(12) Patent Application Publication
(10) Pub. No.: US 2010/0015712 A1
(43) Pub. Date: Jan. 21, 2010

(54) SIDE POPULATION CELLS ORIGINATED FROM HUMAN AMNION AND THEIR USES

(76) Inventors: Norio SAKURAGAWA, Tokyo (JP); Yasunobu Yokoyama, Tokyo (JP)

Correspondence Address: BIRCH STEWART KOLASCH & BIRCH, PO BOX 747, FALLS CHURCH, VA, 22040-0747, US

(21) Appl. No.: 12/499,638

(22) Filed: Jul. 8, 2009

Related U.S. Application Data
(62) Division of application No. 10/919,310, filed on Aug. 17, 2004.

(30) Foreign Application Priority Data
Oct. 28, 2003 (JP) 2003-367258

(51) Int. Cl. C12N 5/04; A61K 48/00

(52) U.S. Cl. 435/378; 435/366; 424/93.7

(57) ABSTRACT

Cells which may be differentiated at least into nerve cells, which are useful for therapies of brain metabolic diseases, are disclosed. The cells are side population cell separated from human amniotic mesenchymal cell layer, in which expressions of Oct-4 gene, Sox-2 gene and Rex-1 gene are observed by RT-PCR, and which are vimentin-positive and CK19-positive in immunocytostaining.

This application is a Divisional of co-pending application Ser. No. 10/919,310 filed on Aug. 17, 2004, and for which priority is claimed under 35 U.S.C. § 120. application Ser. No. 10/919,310 claims priority of Application No. 2003-367258 filed in Japan on Oct. 28, 2003, under 35 U.S.C. § 119, the entire contents of all are hereby incorporated by reference into the present application.

### BACKGROUND OF THE INVENTION

I. Field of the Invention

The present invention relates to cells separated from human amnion. The cells according to the present invention are useful as sources of the substances produced by nerve cells and as drug delivery systems of substances produced by nerve cells when transplanted to the brain of a patient suffering from an intractable nervous disease such as Parkinson's disease or a metabolic nervous disease. Further, since the cells according to the present invention produce specific enzymes, they are useful for therapies of metabolic diseases such as lysosomal disease.

II. Description of the Related Art

Multifunctional stem cells are undifferentiated cells which can differentiate into cells constituting various tissues, which are important in the fields of organ reconstruction and tissue engineering. As the stem cells, myeloid stem cells obtained from bone marrow and cord blood stem cells are known. However, these stem cells have problems in that they are not supplied stably. It was reported that a large amount of multifunctional stem cells may be recovered from human placenta. However, since placenta is originated from mother, when transplanting the cells differentiated from the stem cells originated from placenta, the compatibility of the cells must be checked in order to prevent rejection, and the cells cannot be transplanted to the patient who is not compatible with the cells, which is problematic.

On the other hand, as a method for purifying stem cells, a method using a fluorescent dye called Hoechst 33342 was reported (Goodell, M. A. et al., J. Exp.

Med., 183:1797-1806, 1996). Hoechst 33342 is a fluorescent dye which is incorporated into the nucleotide sequence of “AT” in DNA. Since Hoechst 33342 has a high cell membrane permeability, cells may be stained keeping the cells alive. Therefore, Hoechst 33342 has been used for studying cell cycle of live cells. Upon being irradiated with ultraviolet light, Hoechst 33342 emits two types of fluorescences having wavelengths of 450 nm and 675 nm. Goodell et al. stained mouse bone marrow cells with Hoechst 33342 and subjected the stained cells to fluorescence activated cell sorter (FACS). They carried out two-dimensional analysis taking the fluorescence intensity at 675 mu along the abscissa and taking the fluorescence intensity at 450 mu along the ordinate. As a result, they found that there was a cell population at the lower left area of the coordinate, that is, at the area where the fluorescence intensities of both wavelengths at 450 mu and 675 nm were low. They named the cell population “side population cells” (hereinafter also referred to as “SP cells”). They also revealed that SP cells contain hematopoietic stem cells at a high frequency. By subsequent studies, it was found that SP cells exist also in human, simian, swine, canine, zebrafish and the like in addition to mouse, and that SP cells are also contained in the liver and muscle in addition to bone marrow (Experimental Medicine, Vol.19, No.15 (Special Number), 2001, pp.68-73).

It is thought that SP cells are not well stained with Hoechst 33342 because their abilities to excrete Hoechst 33342 are high, and that Hoechst 33342 is excreted to the outside of the cells by pump-like molecules represented by MDR molecules which are protein encoded by MDR (multi drug resistance gene). It is thought that since MDR is much expressed in stem cells, Hoechst 33342 is excreted outside of the stem cells. Thus, it has been suggested that Hoechst 33342-excreting ability is a characteristic common to stem cells (Experimental Medicine, supra). The SP cell fraction is completely diminished by adding verapamil which is a functional inhibitor of MDR molecule (Procedures of 117th Symposium of The Japanese Association of Medical Sciences, pp.67-74 (August, 2000)).

On the other hand, lysosomal disease is known in which various lysosomal enzymes are deficient. By the lysosomal disease, nervous system such as brain is likely to be disordered, and progressive decrease of intelligence, arrested development and convulsion are caused. As a therapy of lysosomal disease, supplementation of the deficient enzymes is employed. However, by this therapy, it is necessary to continuously supplement the deficient enzymes. There are no radical therapeutic method for lysosomal disease.

### SUMMARY OF THE INVENTION

An object of the present invention is to provide a stem cell which can be supplied stably and which does not have the problem of compatibility when transplanted. Another object of the present invention is to provide a cell useful for therapies of metabolic diseases such as lysosomal disease.

The present inventors intensively studied to discover that SP cells exist in human amniotic mesenchymal cell (hereinafter also referred to as “HAMC”) layer 20 and in human amniotic epithelial cell (hereinafter also referred to as “HAEC”) layer, and that the SP cells express some markers of stem cells, thereby completing the present invention. The present inventors also discovered that the SP cells obtained from human amniotic mesenchymal cell layer produce a number of enzymes such as β-galactosidase and β-glucosidase, that the SP cells are capable of being transplanted to the brain and that the SP cells are useful as cells to be transplanted and useful for therapies of brain metabolic diseases.

That is, the present invention provides a side population cell separated from human amniotic mesenchymal cell layer, in which expressions of Oct-4 gene, Sox-2 gene and Rex-1 gene are observed by RT-PCR, and which is vimentin-positive and CK19-positive in immunocytostaining. The present invention also provides an assemblage of side population cells separated from human amniotic mesenchymal cell layer, comprising cells which are major histocompatibility antigen class II-negative and major histocompatibility antigen class I-negative, and cells which are major histocompatibility antigen class II-negative and major histocompatibility antigen class I-positive. The present invention further provides a method for transplantation comprising transplanting side population cells separated from human amniotic mesenchymal cell layer. The present invention still further provides a method for therapy of a brain metabolic disease, comprising administering an effective amount of side population cells separated from human amniotic mesenchymal cell layer. The present invention still further provides a method for therapy of β-galactosidase-deficiency and/or β-glucosidase-deficiency, comprising administering an effective amount of side population cells separated from human amniotic mesenchymal cell layer.

By the present invention, it was first discovered that SP cells exist in human amniotic mesenchymal cell layer and in human mesenchymal epithelial cell layer, the SP cells were first separated, and it was discovered that the SP cells are stem cells. Since the SP cells originated from HAMC layer according to the present invention can be differentiated at least into nerve cells, they are useful as a source of the substances produced by nerve cells, and may be used as a drug delivery system of the substances produced by nerve cells by being transplanted into the brain of a patient suffering from an intractable nervous disease such as Parkinson's disease or a 25 metabolic nervous disease. Further, the SP cells originated from HAMC layer according to the present invention, which are HLA class I1-negative and HLA class I-negative do not induce immunological rejection when transplanted.

The SP cells separated from HAMC layer can be transplanted to the brain as concretely described in the Examples hereinbelow described, and produce various lysosomal enzymes. Therefore, by transplanting the SP cells according to the present invention into the brain, brain metabolic diseases such as lysosomal disease may be cured.

### BRIEF DESCRIPTION OF THE DRAWING

FIG. 1 shows the results of FACS of the cells separated from HAMC layer or from HAEC layer, which were obtained in Examples of the present invention.

### DETAILED DESCRIPTION OF THE PREFERRED EMBODIMENT

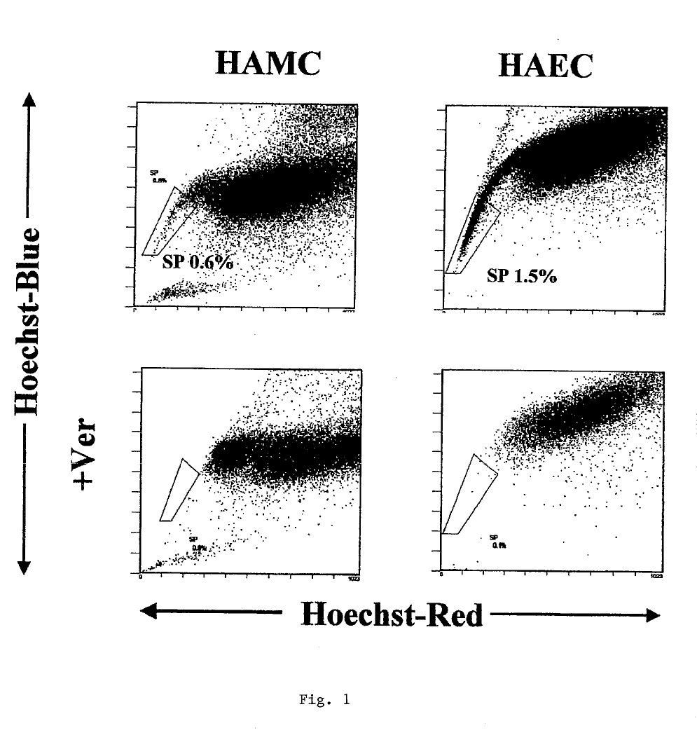

The SP cells according to the present invention are obtained from HAMC layer by the SP cell-separating method which per se is known in the art. That is, as will be concretely described in Examples below, HASAIDC layer is collected and dispersed into single cells. The cells are stained with Hoechst 33342 and the stained cells are subjected to FACS under UV irradiation. Each cell is then plotted in a coordinate taking the fluorescence intensity at a wavelength of 675 nm along the abscissa and taking the fluorescence intensity at a wavelength of 450 nm along the ordinate. In this coordinate, the cells plotted in the lower left area, that is, the cells plotted in the area where the fluorescence intensities of both wavelengths at 450 nm and 675 nm are low, that is, the cells plotted in the area protruded like a horn from the main cell population, are the SP cells. FIG. 1 shows the above-mentioned coordinates in which the cells separated from HAMC layer or HAEC layer were plotted. In each coordinate, the cells plotted in the area encircled by the tetragonal solid line are the SP cells. The SP cells can be recovered by an ordinary method using a FACS apparatus. In the present specification, the term “SP cell(s)” also means the progeny cell(s) obtained by subculturing the SP cell(s), which progeny cell(s) exhibit(s) the properties of the SP cells . In cases where an assemblage of cells is to be specifically referred to, the term “cell assemblage” or “assemblage of cells” may also be used.

The SP cells originated from HAMC layer according to the present invention express Oct-4 gene (POU5 gene) as detected by RT-PCR. It is known that Oct-4 gene is a master gene (differentiation-inhibiting gene) which serves to retain the cell in undifferentiated state, which is expressed only in undifferentiated cell lines that are capable of being cultured in vitro, such as ES cells, EC cells and EG cells (Nichols, J et al.: Cell, 95, 379-391(1998)). The SP cells originated from HAMC layer according to the present invention also express Sox-2 gene and Rex-1 gene. These genes are located downstream of Oct-4 gene. Thus, the SP cells originated from HAMC layer according to the present invention are multipotential stem cells. The cells are vimentin-positive, which is an antigen marker of mesenchymal cells, and the cells are CK-19 positive, which is an antigen marker of epithelial cells.

In the SP cells as separated from HAMC layer, both the cells which are major compatibility antigen (HLA) class II-negative and class I-negative, and the cells which are HLA class II-negative and class I-positive are included, and these types of cells exist in mixed state. Since class I-negative cells are preferred when the cells are used for transplantation, it is preferred to collect only the class I-negative cells and use the cells for transplantation. The present invention also provides an SP cell which is included in the SP cell population originated from HAMC layer according to the present invention and which is HLA-Class I-negative. The class I-negative cells may easily be recovered by collecting the cells which are not stained with HLA-Class I antibody. This collection may be attained by a conventional method using a commercially available flow cytometry apparatus, or by negative selection using magnetic beads.

The SP cell according to the present invention can be subcultured under the following conditions. Culturing conditions: in 10% fetal bovine serum (FBS)-containing DMEM/F-12 (1:1) culture medium supplemented with 10 ng/ml of human leukocyte-inhibition factor (hLIF, Alomone Labo, Israel) and 0.2 mM 2-mercaptoethanol (2-ME, Sigma), at 37° C. on a collagen-coated dish in 5% CO₂ incubator. The cells after 20 passages retain the above-mentioned characteristics of SP cells and express the above-mentioned markers, so that the cells have the growth abilities as stem cells.

Since the SP cells originated from HAMC layer according to the present invention express at least a marker of nerve stem cells, they may be used at least as the nerve stem cells. Thus, the cells according to the present invention are useful at least as a source of the substances produced by nerve cells, and may be used as a drug delivery system of the substances produced by nerve cells by being transplanted into the brain of a patient suffering from an intractable nervous disease such as Parkinson's disease or a metabolic nervous disease.

The SP cells separated from HAMC layer are thought to be a mixture of a plurality of types of cells. However, since the cells can be transplanted as they are without further selection based on the expressions of the above-mentioned genes, based on the results of immunostaining or based on the expression of the major histocompatibility antigen, and since the cells produce the lysosomal enzymes, the cells are useful for therapies of metabolic diseases such as lysosomal disease. Thus, the present invention also provides an assemblage of side population cells separated from human amniotic mesenchymal cell layer, comprising cells which are major histocompatibility antigen class II-negative and major histocompatibility antigen class I-negative, and cells which are major histocompatibility antigen class II-negative and major histocompatibility antigen class I-positive. The SP cell assemblage may also be cultured under the same conditions as the SP cells according to the present invention, which conditions are described above.

As will be concretely described in Examples below, it was confirmed that the SP cells separated from HAMC layer can be transplanted to the brain. Therefore, the present invention also provides a method for transplantation comprising transplanting side population cells separated from HAMC layer. Further, as will be concretely described in Examples below, the SP cells separated from HAMC layer produce a number of lysosomal enzymes, such as β-galactosidase and β-glucosidase. Lysosomal disease favorably occurs in brain and is a representative brain metabolic disease. Thus, the present invention also provides a method for therapy of a brain metabolic disease, comprising administering an effective amount of side population cells separated from human amniotic mesenchymal cell layer. Further, since the SP cells separated from HAMC layer produce β-galactosidase and β-glucosidase in especially larger amounts than other cells, they may be used for the therapies of, among metabolic diseases, the deficiencies of β-galactosidase and/or β-glucosidase.

Transplantation of the cells into an organ such as brain may be easily carried out by injecting a suspension of the cells in a buffer such as phosphate buffer. In cases where the cells are planted to the brain, a small hole is opened through the skull and the suspension may be injected to the brain. Although dose (effective amount) of the cells to be transplanted may be selected depending on the type of the disease, conditions of the patient or the like, the number of cells to be transplanted may usually be about 10³ to 10⁷ cells, preferably 10⁴ to 10⁶ cells. The concentration of the cell suspension to be injected into the brain is not restricted, and may preferably be about 2.0×10⁴ to 2.0×10⁵ cells/μL.

The present invention will now be described by way of examples thereof. It should be noted that the Examples are presented for the illustration purpose only and should not be interpreted in any restrictive way. In the following Examples, all “%” are by weight unless otherwise specified or unless otherwise apparent from the context.

### EXAMPLE 1

### Separation of SP Cells

### 1. Separation of Amniotic Cells and Primary Culture Thereof

(1) After informed consent, HAMC layer and HAEC layer were separated by being peeled off from the chorionic membrane layer in a placenta after scheduled Caesarean operation.
(2) The layers were treated with 0.25% trypsin solution/1.3 mM EDTA at 37° C. for 15 minutes. This operation was repeated 4 times. The trypsin solution fraction was centrifuged to collect the cells, and the cells were washed three times with phosphate buffer (PBS) to obtain HAEC.
(3) After washing the non-digested fraction with PBS, the cells were treated with a mixed enzyme (0.01% papain, 1 mg/ml of collagenase, 0.01% DNase and 0.1% neutral protease) at 37° C. for 1 hour under shaking.
(4) The resultant was centrifuged at 2000 rpm for 10 minutes, and the obtained precipitate was washed three times with PBS, followed by filtering the cells through a filter with an average pore size of 40 μm to obtain mixed enzyme-treated fraction, that is, HAMC.
(5) The cells in each of the fractions were primary cultured in 10% fetal bovine serum (FBS)-containing DMEM/F-12 (1:1) culture medium supplemented with 10 ng/ml of human leukocyte-inhibition factor (HLIF, Alomone Labo, Israel) and 0.2 mM of 2-mercaptoethanol (2-ME, Sigma), at 37° C. on a collagen-coated dish in 5% CO₂ incubator.

### 2. Detection of SP Cells

(1) The primary cultured cells were treated with 0.125% trypsin solution/1.3 mM EDTA at 37° C. for 10 minutes, and the resultant was centrifuged at 2000 rpm for 5 minutes, followed by washing the precipitate twice with PBS.
(2) The resulting cells were suspended in HBSS+ (Hanks Balanced Salt Solution, Gibco) supplemented with 2% FCS and 10 mM HEPES buffer, Gibco), and the number of nucleated cells was counted.
(3) The cells were collected by centrifugation and were suspended in DMEM+ (DMEM, Gibco supplemented with 2% FCS and 10 mM HEPES buffer, Gibco) to a final concentration of 1×10⁶ cells/mL, and the cells were well mixed.
(4) Hoechst 33342 was added to the suspension to a final concentration of 5 μg/mL.
(5) The cells were well mixed and then incubated in a water bath at 37° C. for 120 minutes.
(6) After the incubation, the cells were filtered through a filter having an average pore size of 70 μm and then centrifuged under cooled condition. The cells were then resuspended in cold DMEM+.
(7) While keeping the cells at 4° C., propidium iodide (PI) was added to a final concentration of 2 μg/ml.
(8) To the cells prepared by the above-described operations (1) to (3), Hoechst 33342 (final concentration of 5 μg/mL) and 50 μM verapamil were added, and the above-described operations (4) and (5) were repeated to obtain a sample.
(9) Using EPICS ALTRA HyPerSort (Beckman Coulter), Hoechst 33342 was excited by UV laser at 350 nm, and Hoechst Blue/350 nm and Hoechst Red/675 nm were detected.

### 3. Separation of SP Cells

(1) Ten thousand cells in the SP area were sorted and separated.
(2) The separated cells were cultured in 10% fetal bovine serum (FBS)-containing DMEM/F-12 (1:1) culture medium supplemented with 10 ng/ml of human leukocyte-inhibition factor (hLIF, Alomone Labo, Israel) and 0.2 MM of 2-mercaptoethanol (2-ME, Sigma), at 37° C. on a collagen-coated dish in 5% CO₂ incubator.
(3) The cultured cells were sown at a density of 1.2×10⁵ to 2.8×10⁵ cells/cm², and were subcultured at 3-4 days interval.

The results of the FACS are shown in FIG. 1. As mentioned above, the area encircled by the tetragonal solid line is the SP cell area. About 0.6% of HAMC, and about 1.5% of HAEC were SP cells. The SP cell fraction was completely disappeared by adding verapamil which is a functional inhibitor of MDR molecules. Therefore, it was confirmed that the cells included in the SP area were SP cells.

### EXAMPLE 2

### Analysis of Expressions of Genes by RT-PCR

(1) Total RNAs were extracted from cultured cells after 10 passages using High Pure RNA Isolation Kit (Roche).
(2) Using M-MuLV Reverse Transcriptase (Roche), cDNAs were synthesized from the obtained total RNAs. The conditions for the synthesis of cDNAs were as follows:

| | | |
|---|---|---|
| 5 x Incubation buffer | 4 | μl |
| 10 mM dNTP mix. | 2 | μl |
| 0.1 M DTT | 2 | μl |
| Random primer | 1 | μl |
| (or Oligo dT(18) primer) | 1 | μl |
| RNase inhibitor | 0.5 | μl |
| DEPC treated water | 5 | μl |
| Reverse Transcriptase | 0.5 | μl |
| RNA | 5 | μl |
| Total | 20 | μl |

PCR was carried out under the following conditions:

| | | |
|---|---|---|
| 10 x reaction buffer | 5 | μl |
| 2.5 mM dNTP mix. | 5 | μl |
| 50 μM forward primer | 1 | μl |
| 50 μM reverse primer | 1 | μl |
| Distilled water | 32.5 | μl |
| Taq DNA polymerase | 0.5 | μl |
| cDNA | 5 | μl |
| Total | 50 | μl |

The primers used for the PCR for amplification of the respective genes had the following nucleotide sequences: The annealing temperatures are also shown.

OCT-4 gene (annealing temperature: 62° C.)
5′-ctt gct gca gaa gtg ggt gga gga a-3′
5′-ctg cag tgt ggg ttt cgg gca-3′
nestin gene (annealing temperature: 58° C.)
5′-gag agg gag gac aaa gtc cc-3′
5′-tcc ctc aga gac tag cgc at-3′
Musashi-1 gene (annealing temperature: 60° C.)
5′-gaa tgg acg cct tca tgc tg-3′
5′-cgc tga tgt aac tgc tga cc-3′
Sox-2 gene (annealing temperature: 60° C.)
5′-ccc ccg gcg gca ata gca-3′
5′-tcg gcg ccg ggg aga tac at-3′
Rex-1 gene (annealing temperature: 56° C.)
5′-gcg tac gca aat taa agt cca ga-3′
5′-cag cat cct aaa cag ctc gca gaa t-3′

As a result of RT-PCR, the SP cells originated from HAMC layer were Oct-4-positive, nestin-positive, Sox-2-positive and Rex-1-positive. As for Musashi-1, the cells at the time of completion of 6 passages were negative and the cells at the time of completion of 11 passages were positive. Since Oct-4 gene is a master gene which selves to retain the cell in undifferentiated state, which is expressed only in undifferentiated cell lines that are capable of being cultured in vitro, such as ES cells, EC cells and EG cells, it was proved that the SP cells originated from HAMC layer are multipotential stem cells. Further, since nestin is a marker of nerve stem cells, it was proved that the cells according to the present invention are at least nerve stem cells, that is, the cells according to the present invention are capable of being differentiated at least into nerve cells. It has been reported that among the genes located downstream of Oct-4 gene, Rex-1 is activated by the cooperation of Sox-2 and Rox-1 (cofactor). Thus, in the SP cells originated from HAMC layer according to the present invention, the above-mentioned results indicate that the genes serve to keep the cells in the undifferentiated state.

### EXAMPLE 3

### Immunostaining

(1) Cultured cells were fixed with 4% paraformaldehyde for 1 minute and the fixed cells were incubated with a primary antibody at room temperature for 2 hours.
(2) The resultant was then incubated with a secondary antibody diluted with 0.3% Triton X100 (trademark) for 2 hours.
(3) The immunoblotted cells were observed with a fluorescence microscope and confocal image observed with a confocal laser scanning microscope was analyzed.
(4) The primary antibodies used were anti-human nestin polyclonal antibody, anti-human musashi-1 monoclonal antibody, monoclonal antibodies to CK19 (Santa Cruz), vimentin (PROGEN), CD4 (IMMUNOTECH), CD8 (IMMUNOTECH), CD13 (IMMUNOTECH), CD15 (IMMUNOTECH), CD29(IMMUNOTECH), CD34(IMMUNOTECH), CD38 (IMMUNOTECH), CD43 (IMMUNOTECH), CD44 (IMMUNOTECH), CD45 (IMMUNOTECH), CD49b (IMMUNOTECH), CD50 (IMMUNOTECH), CD56 (IMMUNOTECH), Thy-I (IMMUNOTECH), CD106 (IMMUNOTECH), c-kit (IMMUNOTECH), HLA-DR (Ancell), HLA ClassI, Flt-1 (SANT CRUZ), and AFP (DAKO). Secondary antibodies used were anti-rabbit IgG Rhodamine (1:100, Chemicon) and anti-rabbit IgG FITC (ZYMED). Using these primary antibodies and secondary antibodies, the cell markers were checked by a conventional method.

As a result, the SP cells originated from HAMC layer according to the present invention are vimentin-positive and CK19-positive, and weakly positive for CD29, CD44, Flt-1 and nestin. The cells were HLA Class II-negative. The cells included HLA Class I-negative cells and HLA Class I-positive cells. These two types of cells existed under mixed state. The cells were negative for the other cell markers.

### EXAMPLE 4

Transplantation of SP Cells into Brain

After informed consent, amnion was peeled off from the supplied placental tissue, and SP cells were separated as in Example 1. The obtained SP cells were stained with PKH26 dye so as to label the cells with the dye. That is, to 500 μL of cell suspension in a buffer solution, PKH26 dye was added and the resulting suspension was left to stand at room temperature for 5 minutes. Thereafter, 500 μL of serum was added thereto, followed by washing the cells with PBS. About 5×10⁴ to 2×10⁵ cells after the staining were transplanted to the brain (hippocampus) of a rat (Wistar Rat, 8 weeks old, male). This was carried out by anesthetizing the rat by injection of pentobarbital sodium solution, opening a small hole through the skull at one side, and by injecting the cells into the brain. One week after the transplantation of the cells, the rat was anesthetized by ether. After performing perfusion fixation with 4% paraformaldehyde, the brain was taken out and the tissue of the brain was fixed, followed by preparation of sections. The sections were observed with a fluorescence microscope.

### Results

The SP cells transplanted into the brain migrated along the callous, and migration in a wide range up to the opposite side of the brain was observed. A part of the SP cells taken by the dentate gyrus have changed their morphology from nearly circular shape to the nerve cell-like shape having projections extending to the ambient tissue. Thus, since the SP cells were taken by the brain and changed their morphology to nerve cell-like shape, it was confirmed that the SP cells according to the present invention are capable of being transplanted to the brain.

### EXAMPLE 5

### Production of Lysosomal Enzymes

After informed consent, amnion was peeled off from the supplied placental tissue, and SP cells were separated as in Example 1. The obtained SP cells were checked for the production of the 17 enzymes shown in Table 1.

TABLE 1
Enzymes and Reagents

| | | MPS | | |
|---|---|---|---|---|
| | Enzyme | Type | Main Reagent(Substrate) | Manufacturer |
| 1 | α-L-iduronidase | I | 4MU-α-L-iduronide | Calbio Chem |
| | | | cyclohexylammonium salt | |
| 2 | iduronate-2-sulfatase | II | 4MU-α-iduronide-2-sulfate | Erasmus University |
| | | | | (Netherlands) |
| 3 | heparan-N-sulfatase | III A | 4MU-α-D-N-sulfoglucosaminide | Erasmus University |
| | | | | (Netherlands) |
| 4 | α-NAc glucosaminidase | III B | pNP-α-N-acetyl-D-glucosaminide | Sigma |
| 5 | GlcNAc transferase | III C | 4MU-β-D-glucosamine | Erasmus University |
| | | | | (Netherlands) |
| 6 | GalNAc6 sulfatase | IV A | 4MU-β-D-galactoside-6-sulfate | Erasmus University |
| | | | | (Netherlands) |
| 7 | allylsulfatase B | VI | 4NC-sulfate dipotassium salt | Sigma |
| 8 | α-glucosidase | — | 4MU-α-D-glucoside | ICN Biomedicals, Inc |
| 9 | β-glucosidase | — | 4MU-β-D-glucoside | Nacalai Tesque |
| 10 | α-galactosidase A | — | 4MU-α-D-galactoside, | Nacalai Tesque |
| | | | N-acetyl-D galactosamine | |
| 11 | β-galactosidase | IV B | 4MU-β-D-galactoside | Nacalai Tesque |
| 12 | α-mannosidase | — | 4MU-α-D-mannopyranoside | Calbio Chem |
| 13 | α-fucosidase | — | 4MU-α-L-fucoside | ICN Biomedicals, Inc |
| 14 | β-glucuronidase | VII | 4MU-β-D-glucuronide | Nacalai Tesque |
| 15 | β-hexosaminidase | — | 4MU-β-D-glucosaminide | Nacalai Tesque |
| 16 | β-hexosaminidase A | — | 4MU-β-D-N-acetyl-glucosamine-6- | Calbio Chem |
| | | | sulfate | |
| 17 | allylsulfatase A | — | p-nitrocatecol sulfate · dipotassium | Nacalai Tesque |

### (1) Preparation of Enzyme Samples

To the cultured cell pellet stored in frozen condition, purified water was added to attain a protein concentration of 0.5 to 1 mg/mL, and the resulting mixture was subjected to ultrasonication with a ultrasonic homogenizer to obtain an enzyme sample.

### (2) Measurement of Protein Concentration

The total proteins in the enzyme solution were quantified by the Pyrogallol Red method (Micro TP Test Wako (Wako Pure Chemical Industries, Ltd)).

### (3) Measurements of Enzyme Activities

### Enzyme 1

The substrate was added to the enzyme sample, and the resultant was incubated at 37° C. for 1 hour, followed by adding the stop solution.

### Enzyme 2

The substrate was added to the enzyme sample, and the resultant was incubated at 37° C. for 4 hours. Thereafter, LEBT was added, and the resultant was incubated at 37° C. for another 24 hours, followed by adding the stop solution.

### Enzyme 3

The substrate was added to the enzyme sample, and the resultant was incubated at 37° C. for 17 hours. Thereafter, β-glucosidase was added, and the resultant was incubated at 37° C. for another 24 hours, followed by adding the stop solution.

### Enzyme 5

The substrate was added to the enzyme sample, and the resultant was incubated at 37° C. for 17 hours, followed by adding the stop solution.

### Enzyme 6

The substrate was added to the enzyme sample, and the resultant was incubated at 37° C. for 17 hours. Thereafter, β-glucosidase was added, and the resultant was incubated at 37° C. for another 24 hours, followed by adding the stop solution.

In cases of Enzymes 1 to 3, 5 and 6, after adding the stop solution, the fluorescence intensities were measured with a plate reader (EX (excitation): 365 nm, EM (emission): 450 nm).

### Enzymes 8-16

The substrate was added to the enzyme sample, and the resultant was incubated at 37° C. for 1 hour, followed by adding the stop solution. Thereafter, fluorescence intensities (EX: 365 nm, EM: 450 nm) were measured with a fluorescence analysis system (JASCO CORPORATION).

### Enzymes 7 and 17

The substrate was added to the enzyme sample, and the resultant was incubated at 37° C. for 30 minutes, followed by adding the stop solution. The samples of Enzymes 7 and 9 were incubated at 37° C. for 30 minutes and 90 minutes, respectively, and stop solution was added. Thereafter, absorbance at 515 nm was measured with a spectrophotometer.

### Enzyme 4

The substrate was added to the enzyme sample, and the resultant was incubated at 37° C. for 18 hours, followed by adding the stop solution. Thereafter, the absorbance at 400 nm was measured with a plate reader. (The numbers after the term “Enzyme” are the numbers attached to each enzyme, shown in Table 1).

The results are shown in Table 2. As shown in Table 2, the activities of 7 enzymes (i.e., iduronate-2-sulfatase, heparan-N-sulfatase, 3-glucosidase, β-galactosidase, α-fucosidase, β-hexosaminidase and allylsulfatase A) in the SP cells were more than twice of those in the leukocytes or fibroblast cells. Especially, the activities of β-galactosidase and β-glucosidase were as high as 4.2 times and 2.5 times, respectively, the enzyme activities in the leukocytes or fibroblast cells. Therefore, the SP cells according to the present invention are useful for therapies for deficiencies of these enzymes.

TABLE 2
Results

| | | Enzyme | | | |
|---|---|---|---|---|---|
| | | Activity | | | |
| | Enzyme | in SPC | Reference Data | | Unit |
| 1 | α-L-iduronidase | 164 | 56-201 | (n = 6)f | nmol/mg protein/hr |
| 2 | iduronate-2-sulfatase | 225 | 47-105 | (n = 5)f | nmol/mg protein/4 hr |
| 3 | heparan-N-sulfatase | 107 | 4-52 | (n = 4)f | nmol/mg protein/17 hr |
| 4 | α-NAc glucosaminidase | 94.1 | 79-128 | (n = 4)f | nmol/mg protein/18 hr |
| 5 | GlcNAc transferase | 214 | 60-121 | (n = 5)f | nmol/mg protein/17 hr |
| 6 | GalNAc6 sulfatase | 225 | 146-361 | (n = 5)f | nmol/mg protein/17 hr |
| 7 | allylsulfatase B | 960 | 154-545 | (n = 5) | nmol/mg protein/hr |
| 8 | α-glucosidase | 52.8 | 13.1-46.3 | (*) | nmol/mg protein/hr |
| 9 | β-glucosidase | 140 | 4.1-9.7 | (*) | nmol/mg protein/hr |
| 10 | α-galactosidase A | 83.0 | 49.8-116.4 | (**)f | nmol/mg protein/hr |
| 11 | β-galactosidase | 976 | 37.6-230.1 | (*) | nmol/mg protein/hr |
| 12 | α-mannosidase | 272 | 121.1-345.1 | (*) | nmol/mg protein/hr |
| 13 | α-fucosidase | 200 | 33.1-81.5 | (*)f | nmol/mg protein/hr |
| 14 | β-glucuronidase | 25.5 | 116.4-240.4 | (*) | nmol/mg protein/hr |
| 15 | β-hexosaminidase | 3970 | 401.7-1426.0 | (*) | nmol/mg protein/hr |
| 16 | β-hexosaminidase A | 512 | 251.1-607.4 | (**) | nmol/mg protein/hr |
| 17 | allylsulfatase A | 444 | 109.0-217.2 | (*) | nmol/mg protein/hr |

Footnote for Reference Data
(*) measured value of leukocytes n = 100 (volunteers from employees)
(**) measured value of leukocytes n = 48 (volunteers from employees) The symbol of “f” indicates the measured values of fibroblast cells. SPC: Side Population Cells

1. A method for obtaining an assemblage of side population cells that expresses Oct-4 gene, Sox-2 gene and Rex-₁ gene as assayed by RT-PCR, and is vimentin-positive and CK19-positive as assayed by immunocytostaining, said method comprising:
separating said assemblage of side population cells from human amniotic mesenchymal cell layer.

2. The method according to claim 1, wherein said side population cells is major histocompatibility antigen class II-negative and major histocompatibility antigen class I-positive.

3-7. (canceled)
